(19) United States
(12) Patent Application Publication
(10) Pub. No.: US 2017/0157316 A1
(43) Pub. Date: Jun. 8, 2017

(54) DEFLECTABLE PLUNGER HEAD

(71) Applicant: GE Healthcare AS, Oslo (NO)

(72) Inventor: Martin Monteagle Browne, Oslo (NO)

(21) Appl. No.: 15/327,582

(22) PCT Filed: Jul. 20, 2015
(86) PCT No.: PCT/US2015/041126

§ 371 Date: Jan. 19, 2017

Related U.S. Application Data
(60) Provisional application No. 62/026,908, filed on Jul. 21, 2014.

(51) Int. Cl. A61M 5/00; A61M 5/31; A61M 5/315

(52) U.S. Cl. CPC A61M 5/007; A61M 5/001; A61M 5/31511; A61M 5/3129; A61M 2005/3131

(57) ABSTRACT

The present invention provides a plunger head designed to absorb the increased pressure applied against it during heat sterilization to thereby minimize or prevent plunger head movement during autoclaving. The present invention provides the plunger head as part of a syringe or as part of a kit including a syringe, a plunger rod, and optionally, a pharmaceutical product fluid such as a contrast media. Some pharmaceutically-tolerable gas may be provided in the syringe with the product fluid.

### FIELD OF THE INVENTION

The present invention relates to container used for injectable fluids. More specifically, the present invention is directed to a syringe having a deflectable syringe plunger head.

### BACKGROUND OF THE INVENTION

In the field of injectable solutions, the art often employs syringes or cartridges having an elongate syringe barrel in which is positioned a plunger head. The plunger head slideably moves along an interior surface of the syringe barrel under urging of a plunger or, in the case of cartridges, an injection rod of an automated injector. The syringes, or cartridges, may be provided pre-filled with a pharmaceutical product fluid, including but not limited, to a contrast media. Prior to dispensing, such pre-filled syringes typically undergo a heat sterilization process such as autoclaving. During autoclaving of a pre-filled syringe the temperature varies from typically room temperature up to the sterilization temperature, then after a sterilization hold time, back down to room temperature. Typically, autoclaving results in the contents of the syringe being heated up to and beyond 100° C. Inside the pre-filled syringe there is typically a headspace, filled with air or a gas mixture, and a volume of the pharmaceutical product liquid. When such a confined mixture of gas and liquid is taken through the autoclaving temperature cycle, it is close to impossible to maintain a perfect pressure balance that prevents the movement of the syringe plunger. Thus, as a result of the heating, the syringe plunger head is pushed back from the dispense end of the syringe.

Referring to FIG. 1 and FIG. 2, a typical prior art syringe 1 is shown. Syringe 1 includes an elongate syringe barrel body 2. Syringe barrel body 2 include a first syringe barrel end 3 defining a first syringe barrel aperture 4, an opposed second syringe barrel end 5 defining a second syringe barrel aperture 6, and an elongate cylindrical syringe barrel wall 7 extending between the first end 3 and second end 5. Syringe barrel wall 7 includes opposed outer and inner surfaces 7A and 7B. Inner syringe barrel surface 7B defines an elongate syringe barrel cavity 8 extending in fluid communication between first and second syringe barrel apertures 4 and 6. The syringe barrel 1 comprises a plunger head 10. Plunger head 10 includes opposed first and second ends 14 and 16, respectively, and an elongate cylindrical wall 18 extending therebetween. Cylindrical wall 18 defines an elongate plunger head cavity 20. Second end 16 defines a plunger head aperture 22 in open fluid communication with cavity 20. Plunger head 10 also includes a head wall 24 exending across first end 14 in overlying registry with cavity 20. Plunger head 10 may further include one or more annular grooves 32 defined about the outer cylindrical surface of cylindrical wall 18. Plunger head 10 accepts a first end 70 of an elongate plunger rod 75 into cavity 18 so as to move plunger head 10 within syringe barrel cavity 8. The first end 70 of plunger rod 75 include a transverse planar base 80 including a planar annular surface 82 which engages second end 16 of plunger head 10. The first end of the plunger rod 75 also provides a plunger head mount 84 projecting from surface 82 for insertion into plunger head cavity 20. Head mount 84 typically includes an engaging means such as a helical thread 86 which releasably mates with a matching thread 34 within plunger head 10 to positively engage the plunger head 10.

As described above, a product fluid and some amount of gas may be held within barrel cavity 8 between plunger head 10 and first end 3 of syringe 1. During autoclaving, pressure from the fluid and gas may cause plunger head 10 to move back towards the second end 5 of syringe 1. The distance that the plunger moves back under these conditions thus provides a change of volume holding the product fluid and gas, nominally given by the formula ΔV=Πr²h, where ΔV is the change of volume within the syringe, r is the radius of the cylindrical syringe barrel, and h is the distance that the syringe has moved.

For various reasons there is an objective to avoid movement of a syringe plunger during autoclaving of prefilled syringes. One such reason is that the product subject to autoclaving may get trapped between the syringe barrel and the syringe plunger. Additionally, in the case of oversaturated solutions, which are common in the case of pharmaceutical grade contrast media, the trapped product is liable to dry out, thereby further increasing the concentration until crystallization or solidification occurs. Crystallization or solidification may lead to propagation into the main volume of the syringe and there give rise to crystallization or precipitation, a wholly undesirable result for an injectable product.

The art has seen attempts to prevent this movement of the plunger head including the use of silicon between the plunger head and barrel as well as various counter-pressure strategies applied through autoclaving. The art still needs a means to minimize or avoid movement of a syringe plunger head during autoclaving of prefilled syringes without these additional features being applied to the syringe.

### SUMMARY OF THE INVENTION

In one embodiment, a syringe plunger head is provided. The syringe plunger head includes an elongate plunger head body comprising a first end, an opposed second end, and an elongate cylindrical wall extending between the first and second ends. The cylindrical wall defines an elongate head cavity. The syringe plunger head body further includes a head body aperture defined by the second end of the head body in open fluid communication with the head cavity and a deflectable membrane transversely spanning the first end across the head cavity. The membrane is deflectable between a first position extending longitudinally away from the cylindrical wall and a second position extending towards said cylindrical wall.

In another embodiment, a syringe is provided. The syringe includes an elongate syringe barrel comprising a syringe barrel body. The syringe barrel body comprises a first syringe barrel end defining a first syringe barrel aperture, a second syringe barrel end defining a second syringe barrel aperture, and an elongate cylindrical syringe barrel wall extending between the first and second ends. The syringe barrel wall has opposed outer and inner surfaces, the inner syringe barrel surface defining an elongate syringe barrel cavity extending in fluid communication between the first and second syringe barrel apertures. The syringe further comprises a plunger head. The syringe plunger head includes an elongate plunger head body comprising a first end, an opposed second end, and an elongate cylindrical wall extending between the first and second ends. The cylindrical wall defines an elongate head cavity. The syringe plunger head body further includes a head body aperture defined by the second end of the head body in open fluid communication with the head cavity and a deflectable membrane transversely spanning the first end across the head cavity. The membrane is deflectable between a first position extending longitudinally away from the cylindrical wall and a second position extending towards said cylindrical wall. The plunger head is positioned within the syringe barrel cavity such that the outer surface of the plunger head cylindrical wall slideably engages the inner syringe barrel surface.

In yet another embodiment, a kit is provided. The kit comprises a syringe and a plunger rod. The syringe comprises includes an elongate syringe barrel comprising a syringe barrel body. The syringe barrel body comprises a first syringe barrel end defining a first syringe barrel aperture, a second syringe barrel end defining a second syringe barrel aperture, and an elongate cylindrical syringe barrel wall extending between the first and second ends. The syringe barrel wall has opposed outer and inner surfaces, the inner syringe barrel surface defining an elongate syringe barrel cavity extending in fluid communication between the first and second syringe barrel apertures. The syringe further comprises a plunger head. The syringe plunger head includes an elongate plunger head body comprising a first end, an opposed second end, and an elongate cylindrical wall extending between the first and second ends. The cylindrical wall defines an elongate head cavity. The syringe plunger head body further includes a head body aperture defined by the second end of the head body in open fluid communication with the head cavity and a deflectable membrane transversely spanning the first end across the head cavity. The membrane is deflectable between a first position extending longitudinally away from the cylindrical wall and a second position extending towards said cylindrical wall. The plunger head is positioned within the syringe barrel cavity such that the outer surface of the plunger head cylindrical wall slideably engages the inner syringe barrel surface. The plunger rod comprises a first end, a second end, and an elongate plunger rod body extending therebetween. The first end of the plunger rod is shaped to be inserted into the head cavity of the plunger head.

### BRIEF DESCRIPTION OF THE DRAWINGS

FIG. 1 depicts a syringe plunger of the prior art;

FIG. 2 depicts a partial cut-away view of a syringe with a syringe plunger head of the prior art;

FIG. 3 depicts an embodiment of a plunger of the present invention;

FIG. 4 depicts an embodiment of a deflectable syringe plunger head of the present invention in a non-deflected state;

FIG. 5 depicts an embodiment of the plunger head of FIG. 4 in a deflected state;

FIG. 6A depicts a cross-sectional view of an embodiment of a plunger head of the present invention having a first surface with a sustantially convex shape a first, undeflected configuration;

FIG. 6B depicts a cross-sectional view of an embodiment of the plunger head of FIG. 6A with the first surface having a substantially convex shape in a second, deflected configuration;

FIG. 7A depicts a cross-sectional view of an embodiment of a plunger head of the present invention having a first surface with a sustantially conical shape in a first, undeflected configuration;

FIG. 7B depicts a cross-sectional view of an embodiment of the plunger head of FIG. 7A with the first surface with a substantially conical shape in a second, deflected configuration;

FIG. 8A depicts a cross-sectional view of an embodiment of plunger head of the present invention having a first surface with a sustantially parabolic shape in a first, undeflected configuration;

FIG. 8B depicts a cross-sectional view of an embodiment of the plunger head of FIG. 8A with the first surface with a substantially concial shape in a second, deflected configuration;

FIG. 9A depicts a cross-sectional view of a plunger head of the present invention having a first surface with a sustantially bell shape in both a first, undeflected configuration;

FIG. 9B depicts a cross-sectional view of the plunger head of FIG. 9A with the first surface having a substantially bell shape in a second, deflected configuration; and

FIG. 10 depicts a cross-sectional view of a syringe in accordance with an embodiment of the present invention.

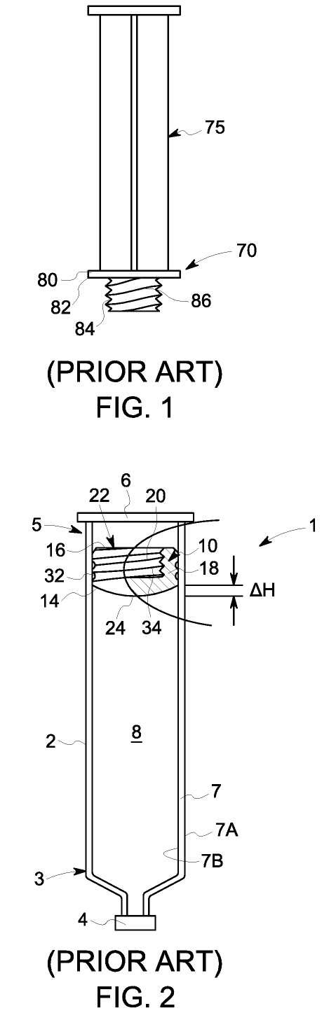

### DETAILED DESCRIPTION OF THE PREFERRED EMBODIMENT

The present invention provides a plunger head designed to absorb the increased pressure applied against it during heat sterilization to thereby minimize or prevent plunger head movement during autoclaving. The present invention provides a plunger having a portion that will deflect so as to provide additional volume which would result during autoclaving, by allowing deformation of the plunger. The forces which would normally cause plunger movement during heat sterilization will instead be substantially, and desirably, entirely absorbed by a flexible portion of the proposed plunger. The deformation of the plunger has a volume change equivalent to the variable volume of the gas, liquid and container volume experienced during heat sterilization. The described invention can thus minimize or eliminate the longitudinal plunger movement, and thereby minimize or eliminate crystallization/precipitation and thus maintain the quality of the injectable product. Desirably, the deflection of the plunger head of the present invention will account for at least 90% of the volume change experienced during heat sterilization, although more desirably, the plunger will account for at least 95% of such volume change, and even more desirably will account for 99-100% (inclusive) of the volume change.

The plunger head of the present invention may be formed from a suitable polymeric material, desirably an elastomeric material suitable for use with pharmaceutical products such as contrast media. Similarly, the plunger head of the present invention may be used with a syringe barrel also made from a suitable material for use with pharmaceutical products. The syringe barrel is desirably formed from a more rigid polymeric material or glass.

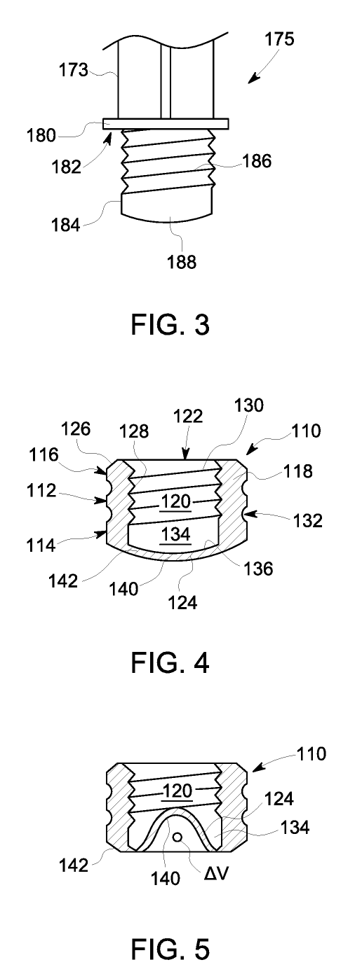

Referring to FIGS. 3-5, the present invention provides a syringe plunger head 110 having an elongate plunger head body 112 which includes a first end 114, an opposed second end 116, and an elongate cylindrical wall 118 extending between first and second ends 114 and 116. Cylindrical wall 118 defines an elongate head cavity 120. Second end 116 defines a head body aperture 122 in open fluid communication with head cavity 120. Plunger head body 112 also includes a deflectable membrane 124 transversely spanning first end 114 across head cavity 120. Membrane 124 is deflectable between a first position extending longitudinally away from cylindrical wall 118 as shown in FIG. 4 and a second position extending towards cylindrical wall 118 as shown, for example, in FIG. 5.

Desirably, membrane 124 deflects an amount which minimizes or eliminates the deflection of plunger head 110 when the contents of a syringe or cartridge containing plunger head 110 are heated during autoclaving. In some embodiements, when in the second position, deflectable membrane 124 extends into head cavity 120. In other embodiments, it is possible that membrane 124 collapses towards cylindrical wall 118 a distance sufficient to increase the volume displaced by the contents during heating under a sterilization or autoclaving process.

Cylindrical wall 118 includes an outer surface 126 and an inner surface 128. Inner surface 128 may include a helical thread 130 which extends therealong from the head body aperture 122 to a termination position adjacent first end 114 or at least sufficiently along inner surface 128 so as to threadingly engage a mating thread 186 on a plunger rod 175. Additionally, outer surface 126 of cylindrical wall 118 defines one or more annular grooves 132 extending thereabout.

Plunger head body 112 desirably defines a head space 134 extending from an inner surface 136 of deflectable membrane 124 towards head cavity 120 so as to be in open fluid communicaiton therewith. Membrane 124 desirably includes an axially central portion 140 and a circumferential portion 142 adjoining first end 114 of cylindrical wall 118, such that central portion 140 extends longitudinally further from the cylindrical wall 118 than circumferential portion 142 when membrane 124 is in the first position. Desirably, central portion 140 extends closer to second end 116 of head body 110 than circumferential portion 142 when membrane 124 is in the second position as shown, for example, in FIG. 5.

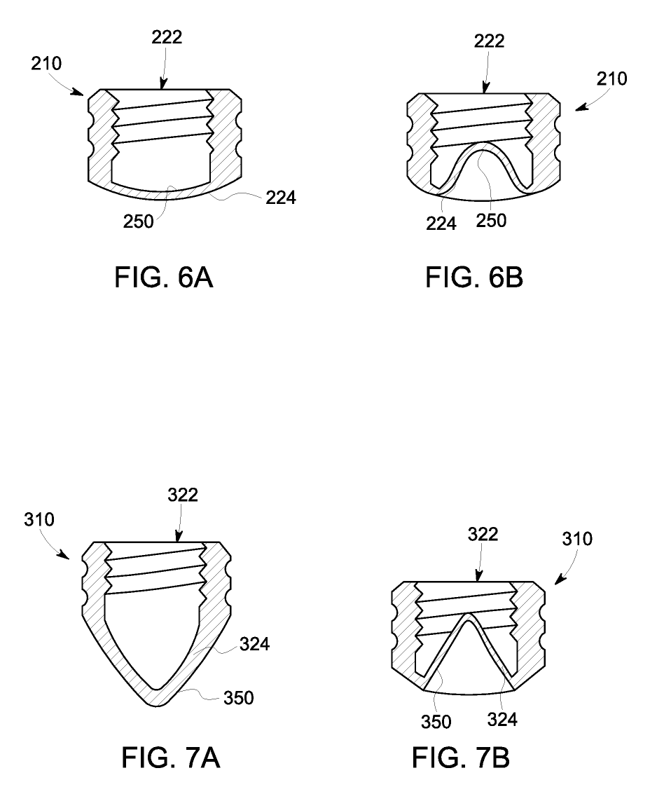

As shown in FIG. 6A, the present invention provides a plunger head 210 having a membrane 224 which includes a first surface 250 facing away from a head body aperture 222, where first surface 222 has a sustantially convex shape in a first, undeflected position. FIG. 6B depicts an embodiment of plunger head 210 having a membrane 224 where the first surface 222 has a substantially convex shape in a second, deflected position.

As shown in FIG. 7A, the present invention provides a plunger head 310 having a membrane 324 which includes a first surface 350 facing away from a head body aperture 322, where first surface 350 has a sustantially conical shape in a first, undeflected position. FIG. 7B depicts an embodiment of plunger head 310 having a membrane 324 where the first surface 322 has a substantially conical shape in a second, deflected position.

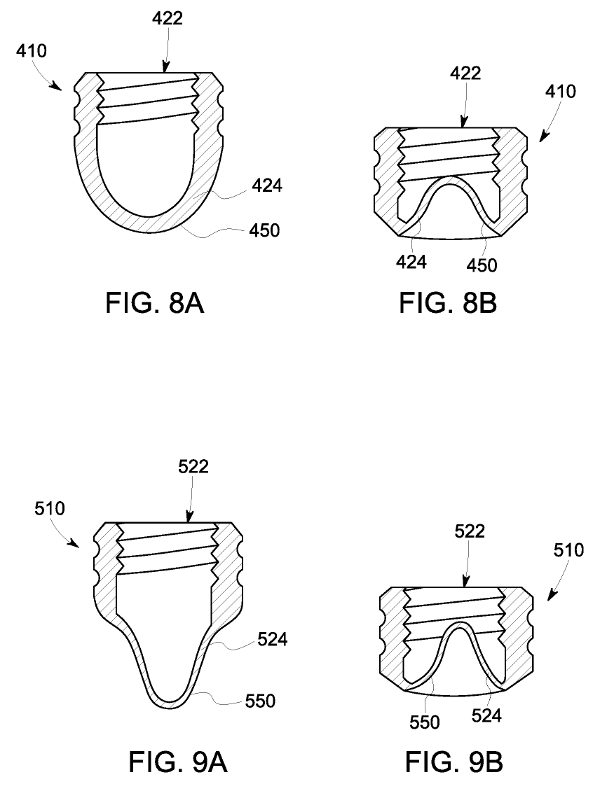

As shown in FIG. 8A, the present invention also provides a plunger head 410 having a membrane 424 which includes a first surface 450 facing away from the head body aperture 422, where first surface 450 has a sustantially parabolic shape in a first, undeflected position. FIG. 8B depicts an embodiment of plunger head 410 having a membrane 424 where the first surface 450 has a substantially parabolic shape in a second, deflected position.

As shown in FIG. 9A, the present invention also provides a plunger head 510 having a membrane 524 which includes a first surface 550 facing away from the head body aperture 522, where first surface 550 has a sustantially bell shape in a first, undeflected position. A ‘bell shape’ is contemplated to have at least three inflection points as seen in cross section and as traced from one point at the cylindrical wall 518 to a diametrically-opposite point on wall 518. FIG. 9B depicts an embodiment of plunger head 510 having a membrane where the first surface 550 has a substantially bell shape in a second, deflected position.

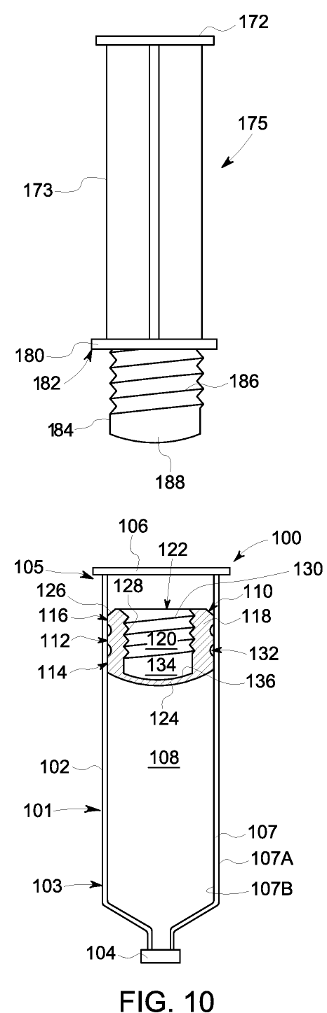

Referring to FIG. 10, the present invention provides a syringe 100 incorporating a plunger head of the present invention. Syringe 100 may also be referred to as a cartridge as the operating principle of the present invention works for any such device which may be autoclaved (or otherwise heat sterilized) while the plunger head does not have a plunger rod fully inserted therein. Syringe 100 includes an elongate syringe barrel 101 having a syringe barrel body 102. Syringe barrel body 102 include a first syringe barrel end 103 defining a first syringe barrel aperture 104, an opposed second syringe barrel end 105 defining a second syringe barrel aperture 106, and an elongate cylindrical syringe barrel wall 107 extending between the first and second ends. Syringe barrel wall 107 includes opposed outer and inner surfaces 107A and 107B. Inner syringe barrel surface 107B defines an elongate syringe barrel cavity 108 extending in fluid communication between first and second syringe barrel apertures 104 and 106.

Syringe 100 includes a plunger head of the present invention, including but not limited to syringe plunger heads 110, 210, 310, 410, and 510 as described herein. The plunger head of the present invention is positioned within syringe barrel cavity 108 such that the outer surface of the plunger head cylindrical wall slideably engages the inner syringe barrel surface. The plunger head of the present invention is slideable under the urging of, for example, either a plunger rod 175 connected thereto or a movable injector rod from an automated injector.

As partially shown in FIGS. 3 and 10, a plunger rod 175 for use with the present invention includes a first end 188, a second end 172, and an elongate plunger rod body 173 extending therebetween. The first end 188 of the plunger rod is shaped to be inserted into the head cavity 120 of a plunger head 110 of the present invention, and is desirably shaped to substantially fill plunger head cavity 120 so as to position membrane 124 in the first, undeflected, position. Alternatively, the shape of first end 188 is desirably configured such that it imparts a shape to membrane 124 that allows for maximum dispensement of the product fluid from syringe 100, thus in some embodiments it may leave some head space 134 between first end 188 of rod 175 and the inner surface 136 of membrane 124. Plunger head 10 removably engages first end 188 of elongate plunger rod 175 into cavity 120 so as to move plunger head 10 within syringe barrel cavity 108. First end 188 of plunger rod 175 include a transverse planar base 180 including a planar annular surface 182 which engages second end 116 of plunger head 110 and assists with moving plunger head 110 towards dispense end 103 of syringe 100. The first end 188 of the plunger rod 175 also provides a plunger head mount 184 projecting from surface 182 for insertion into head cavity 120. Head mount 184 desirably includes an engaging means, such as a helical thread 186, which mates with matching thread 130 within plunger head 110 so as to positively engage plunger head 110. Desirably, plunger rod 175 is removably insertable into head cavity 120 of plunger head 110 and may thus be connected and disconnected from syringe body 101.

The present invention additionally provides a kit including a syringe 100 with a plunger head 110 of the present invention as well as a plunger rod 175 having a first end adapted to be inserted into the head cavity 120 of plunger head 110. The kit of the present invention may further include a pharmaceutical product fluid, such as a contrast media, and a gas, within the syringe 100 such that heat sterilization of the product fluid within the syringe will cause the product fluid and/or gas to act upon plunger head 110 so as to cause membrane 124 to deflect from a first, undeflected position, to a second, deflected, position. The deflection of membrane 124 minimizes, or desirably eliminates, movement of plunger head 110 within syringe 100. The first end 188 of plunger rod 175 is shaped to be inserted into head cavity 120 of plunger head 110. Desirably, the first end 188 of plunger rod 175 is shaped to substantially fill the plunger head cavity so as to position the membrane in the first position. Desirably, plunger rod 175 is removably insertable into head cavity 120 of plunger head 110.

The present invention contemplates that plunger head, syringe, and kit of the present invention are suitable for use with fluid products, particularly pharmaceutical products requiring heat sterilization such as autoclaving. The plunger head, syringe, and kit are suitable for use with injectable contrast media, such as an X-ray contrast media containing ioforminol. The fluid-contacting surfaces of the plunger head, syringe, and kit of the present invention may be formed from suitable materials known in the art for injectable contrast media.

While the particular embodiment of the present invention has been shown and described, it will be apparent to those skilled in the art that changes and modifications may be made without departing from the teachings of the invention. The matter set forth in the foregoing description and accompanying figures is offered by way of illustration only and not as a limitation. The actual scope of the invention is intended to be defined in the following claims when viewed in their proper perspective based on the prior art.

1. A syringe plunger head comprising:
an elongate plunger head body comprising
a first end;
an opposed second end;
an elongate cylindrical wall extending between the first and second ends, the cylindrical wall defining an elongate head cavity;
a head body aperture defined by the second end of the head body in open fluid communication with the head cavity; and
a deflectable membrane transversely spanning the first end across the head cavity, the membrane deflectable between a first position extending longitudinally away from the cylindrical wall and a second position extending towards said cylindrical wall.

2. The plunger head of claim 1, wherein in the second position, the deflectable membrane extends into the head cavity defined by the cylindrical wall.

3. The plunger head of claim 2, wherein the cylindrical wall includes an outer surface and an inner surface, wherein the inner surface includes a helical thread extending from the head body aperture to a termination position adjacent the first end.

4. The plunger head of claim 3, wherein the head body further defines a head space extending from the deflectable membrane to the terminations position of helical thread, the head space in open fluid communication with the head cavity.

5. The plunger head of claim 4, wherein the membrane comprises an axially central portion and a circumferential portion adjoining the first end of the cylindrical wall, wherein the central portion extends longitudinally further from the cylindrical wall than the circumferential portion when the membrane is in the first position.

6. The plunger head of claim 5, wherein the central portion extends closer to the second end of the head body than the circumferential portion when the membrane is in the second position.

7. The plunger of claim 6, wherein the membrane includes a first surface facing away from the head body aperture, the first surface comprising a substantially convex shape.

8. The plunger of claim 6, wherein the membrane includes a first surface facing away from the head body aperture, the first surface comprising a substantially conical shape.

9. The plunger of claim 6, wherein the membrane includes a first surface facing away from the head body aperture, the first surface comprising a substantially parabolic shape.

10. The plunger of claim 6, wherein the membrane includes a first surface facing away from the head body aperture, the first surface comprising a substantially bell shape.

11. The plunger head of claim 10, wherein the outer surface of the cylindrical wall defines at least one annular groove extending thereabout.

12. A syringe comprising:
an elongate syringe barrel comprising a syringe barrel body comprising
a first syringe barrel end defining a first syringe barrel aperture,
a second syringe barrel end defining a second syringe barrel aperture, and
an elongate cylindrical syringe barrel wall extending between the first and second ends, the syringe barrel wall having opposed outer and inner surfaces, wherein the inner syringe barrel surface defines an elongate syringe barrel cavity extending in fluid communication between the first and second syringe barrel apertures; and
the plunger head positioned within the syringe barrel cavity such that the outer surface of the plunger head cylindrical wall slideably engages the inner syringe barrel surface.

13. The syringe of claim 12, further comprising a plunger rod comprising a first end, a second end, and an elongate plunger rod body extending therebetween, the first end of the plunger rod is shaped to be inserted into the head cavity of the plunger head.

14. The syringe of claim 13, wherein the first end of the plunger rod is shaped to substantially fill the plunger head cavity so as to position the membrane in the first position.

15. The syringe of claim 14, wherein the plunger rod is removably insertable into the head cavity of the plunger head.

16. A kit comprising:
a syringe of claim 12; and
a plunger rod comprising a first end, a second end, and an elongate plunger rod body extending therebetween, the first end of the plunger rod is shaped to be inserted into the head cavity of the plunger head.

17. The kit of claim 16, wherein the first end of the plunger rod is shaped to substantially fill the plunger head cavity so as to position the membrane in the first position.

18. The kit of claim 17, wherein the plunger rod is removably insertable into the head cavity of the plunger head.

19. The kit of claim 18, further comprising a product fluid held within the syringe barrel cavity between the plunger head and the first aperture of the syringe barrel.

20. The kit of claim 19, wherein the product fluid is a contrast media.
